(19) United States
(12) Patent Application Publication
(10) Pub. No.: US 2005/0182357 A1
(43) Pub. Date: Aug. 18, 2005

(54) Syringes

(76) Inventors: Richard Andrew Snell, Cambridge (GB); Ben Arlett, Cambridge (GB); Nicholas Andrew Murray Drought, Cambridge (GB); John Vaughan Williams, Cambridge (GB)

Correspondence Address: NEEDLE & ROSENBERG, P.C., SUITE 1000, 999 PEACHTREE STREET, ATLANTA, GA, 30309-3915, US

(21) Appl. No.: 10/522,265

(22) PCT Filed: Jul. 25, 2003
(86) PCT No.: PCT/GB03/03187

(30) Foreign Application Priority Data
Jul. 25, 2002 (GB) 02172856

(51) Int. Cl. A61M 37/00

(52) U.S. Cl. 604/82

(57) ABSTRACT

A two-part syringe comprises two chambers (2,4) each chamber having an associated plunger (6,8) operable to eject fluid therefrom. The plungers (6,8) are interlinked so as selectively to prevent movement of one of said (6,8) plungers in its respective chamber (2,4) dependent upon the position of the other plunger (8,6). Such an arrangement allows lyophilised drugs to be automatically reconstituted within the syringe prior to injection into a patient.

This invention relates to syringes—particularly, although not exclusively, syringes which are suitable for reconstituting lyophilised substances prior to administration.

Many drugs and medicaments are routinely supplied in lyophilised form. This has the advantage of significantly extending the shelf life of the drug or medicament, eliminating the need for the drug to be stored under a careful temperature regime or simply permitting storage of a substance which would otherwise quickly break down. Immediately prior to being required, the lyophilised drug may be reconstituted by mixing it with a suitable diluent.

Conventionally, lyophilised drugs are supplied in glass vials. Glass is used since it is very inert and may therefore safely be used to store a wide variety of substances. Furthermore it has extremely good barrier properties and therefore permits long term storage without contamination etc.

A diluent is either extracted from another glass vial by a syringe with a needle for piercing the rubber septum thereof, or is supplied in a pre-filled syringe. The diluent is then injected into the vial containing the drug by using the needle on the syringe to pierce the rubber septum of the drug vial. Once the drug has been reconstituted, the syringe is used to draw the reconstituted drug out of the vial, ready for injection into a patient.

Clearly the requirement to penetrate a relatively small rubber septum at least once with a needle gives rise to the danger of injury to the user. Furthermore the addition of diluent to the drug is entirely under the control of the user. One problem this can give rise to is that the diluent can be added too quickly. Too high a flow rate of diluent onto the drug can cause it to shear excessively. Lyophilised drugs tend to be delicate and some can be damaged by excessive shearing. A high flow rate may also cause the diluent and drug mixture to undergo undesirable foaming.

Various arrangement have been proposed for allowing lyophilised drugs to be reconstituted inside a device thereby avoiding some of the potential for injury associated with the conventional method of reconstitution. Examples of such arrangements are shown in WO 01/26718, WO 96/29106 and EP-A-1038543. However, all of the previous arrangements suffer from some drawbacks.

It is an object of the invention to provide an improved way of reconstituting drugs and when viewed from a first aspect the invention provides a two-part syringe comprising two chambers, each chamber having an associated plunger operable to eject fluid therefrom, said plungers being interlinked so as selectively to prevent movement of one of said plungers in its respective chamber dependent upon the position of the other plunger.

Thus it will be seen in accordance with the present invention that by suitable design of the interlinking between the two plungers of a two-chamber syringe, the order and extent to which each may be operated can be controlled. By providing a lyophilised drug and diluent respectively in the two chambers, for example, such an arrangement could be used to ensure that diluent is expelled from its chamber and into the chamber containing the lyophilised drug before the reconstituted drug can be expelled. Furthermore it can be arranged that the drug cannot be injected into a patient until the correct quantity of diluent has been used.

Another possible use for an embodiment of the invention might be to ensure that two substances to be administered in conjunction with one another are administered in the correct order.

By interlinking the two plungers in accordance with the invention it may be ensured that, at each stage of a drug preparation and administration procedure, only the correct plunger may be actuated. This prevents any possible accident or misuse of the device meaning that a minimal amount of instruction in how to operate the syringe is required. This potentially opens up the possibilities for drugs to be administered by non-medical staff or by patients themselves, giving increased convenience to patients and saving costs.

The two plungers could be interlinked by any suitable means. For example one plunger could be arranged to release or set a latch, catch or the like preventing operation of the other. Preferably, however, a cam means is associated with the plungers. In a particularly preferred embodiment each plunger comprises a cam track cooperating with a common cam shuttle member comprising followers for each cam track. Preferably such a cam shuttle is constrained to move within a void extending perpendicularly to the plungers. This void could be defined by a separate member or as an integral part of a housing for the syringe.

There are many different patterns of actuation of the two plungers which the interlinking could be configured to allow. The skilled person can therefore select the required pattern depending upon the application for which the syringe is to be used. In preferred embodiments where the syringe is for use in reconstituting and injecting lyophilised drugs, the interlinking is configured to prevent injection of the drug until it has been reconstituted with diluent.

The two plungers could be made identical in appearance. Preferably however, they are different in appearance—for example one may be larger than the other or, preferably, they are different colours. This allows a user easily to identify which should be operated at which stage, thereby avoiding frustration or confusion on the part of a user attempting to operate a plunger which is not intended to be operated at that stage and which, therefore, is prevented from doing so.

The two chambers could be maintained isolated from one another so that their contents are delivered independently subject to the configuration of the plunger interlink. Preferably however, the syringe is arranged such that the two chambers can be placed selectively in fluid communication with one another. This allows, for example, reconstitution of a lyophilised drug within one of the chambers. In a preferred embodiment a communicating conduit is provided which is movable into fluid communication with one or both of the chambers. Most preferably such movement is arranged to breach a seal such as a septum or the like associated with one or both of the chambers. For example, the communicating conduit may comprise one or two needles for breaching said seal or seals. If two needles are provided, they may be arranged to breach the respective seals or the chambers at the same time. Alternatively, they could be arranged to breach the seals in a predetermined order, e.g. by making one longer than the other.

The communicating conduit preferably comprises a tortuous or complex fluid path such that liquid forced therethrough under ordinary manual pressure on one of the plungers is caused to exit the conduit substantially without jetting. Provision of a tortuous path giving a high pressure drop and low fluid exit speed is especially advantageous in the reconstitution of some lyophilised drugs which can be harmed if foaming or shearing occurs.

In particularly preferred embodiments the communicating conduit is provided on a separate support member which is preferably slidable towards or away from the chambers.

By providing the communicating conduit on a separate support member, the transient pathway for the substances can be separated from the part of the apparatus required to provide storage. This allows, in a preferred embodiment, the superior inertness and barrier properties of glass to be used for the storage section whereas the transient section, which is separated from the storage section during storage and thus is not subject to the same stringent inertness and barrier requirements, can be made from plastics. This substantially facilitates its manufacture and easily allows the tortuous path mentioned above to be provided. It also allows, if suited to the particular application, needles to be integrally moulded at the ends of the conduit for breaching seals on the chambers. Alternatively separate e.g. steel needles could be attached to the ends of the conduit for this purpose. Either way, the support member gives another advantage that the user can breach the seals without having to come into contact with a needle since the needles can effectively be embedded within the structure of the syringe.

Furthermore, by providing a separate sliding support member incorporating the communicating conduit, fluidly joining the two previously separated chambers is easily achieved whilst allowing the two chambers to be fabricated, filled and, where appropriate, sterilised separately from one another. An example of why this is advantageous is given by the case of an embodiment in which the two chambers are pre-filled with lyophilised drug and diluent respectively. The diluent chamber and peripheral parts of the device must, in general, be sterilised. However, the sterilisation process can often damage lyophilised drugs. In accordance with the arrangement described above however, the two chambers may be prepared independently of one another and mounted together only in a final step. This facilitates production of such devices.

In fact it will be appreciated that the foregoing arrangements are novel and inventive in their own right and thus when viewed from a second aspect the invention provides a syringe for reconstituting a lyophilised substance comprising a first chamber containing or for containing the lyophilised substance and a second chamber containing or for containing a diluent; the first and second chambers being arranged generally adjacent one another; the syringe further comprising a communicating conduit for fluidly connecting the two chambers, said fluid connection means being selectively engageable with the two chambers.

Preferably the conduit is provided on a support member separate from the two chambers. Most preferably the support is such as to be slidable so as to bring the conduit into and out of engagement with the chambers. It is also preferred that the two chambers have respective associated plungers which are interlinked to prevent activation of one depending upon the position of the other in accordance with the first aspect of the invention. Similarly it is also preferred that the conduit has a tortuous configuration as previously described to prevent foaming or shearing of the drug during reconstitution.

Where in the present application the term “plunger” is used, it should be appreciated that the invention is not limited to any particular form of construction and that in fact any suitable means of reducing volume in the chamber and/or expelling the contents therefrom could be substituted.

A preferred embodiment of the invention will now be described, by way of example only, with reference to the accompanying drawings in which:

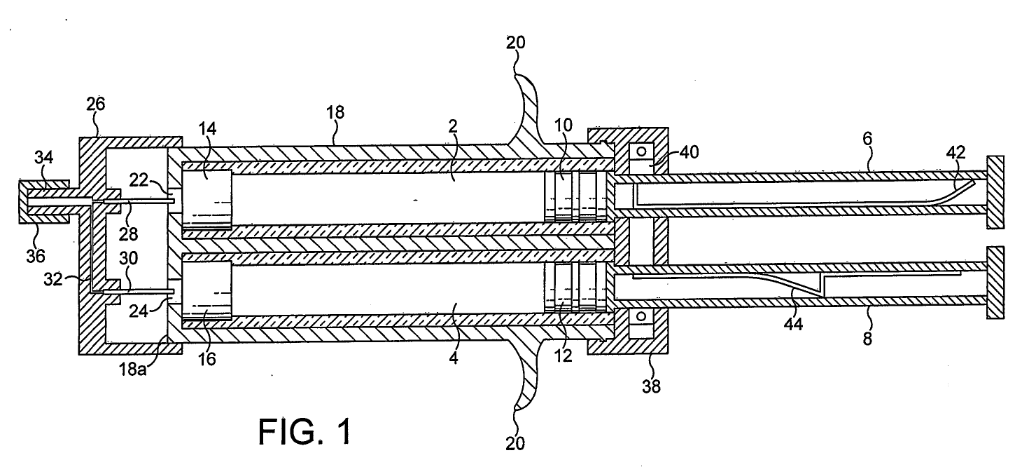

FIG. 1 is a cross-section through a syringe in accordance with the invention in its storage position;

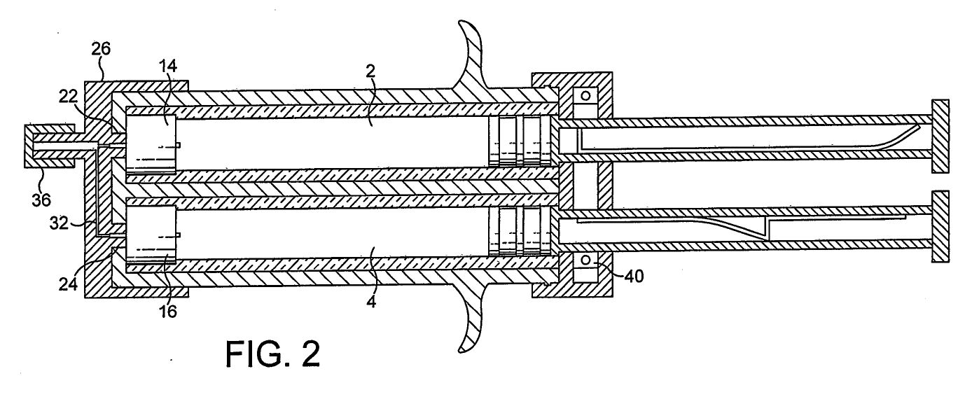

FIG. 2 shows the syringe of FIG. 1 with the septums pierced;

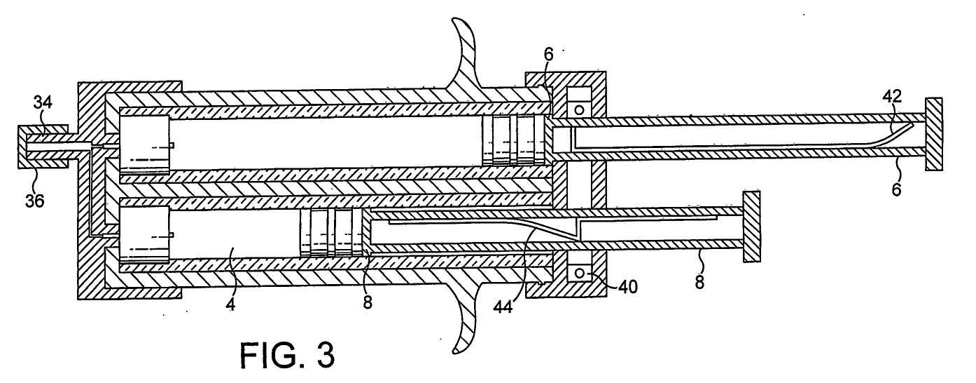

FIG. 3 shows the syringe after reconstitution of a drug;

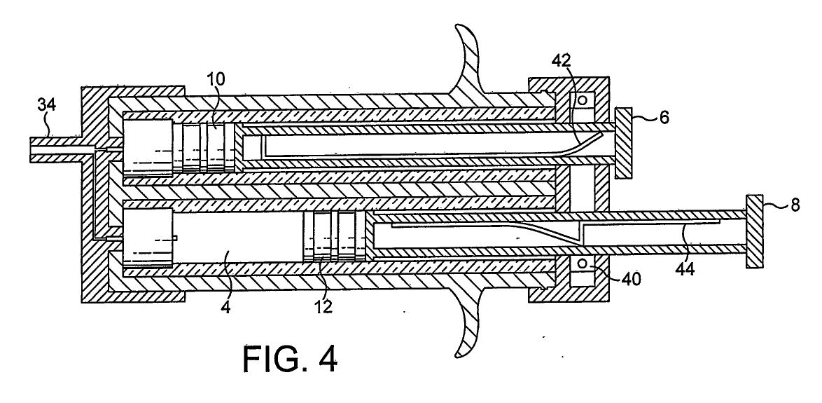

FIG. 4 shows the syringe after purge and injection of the drug; and

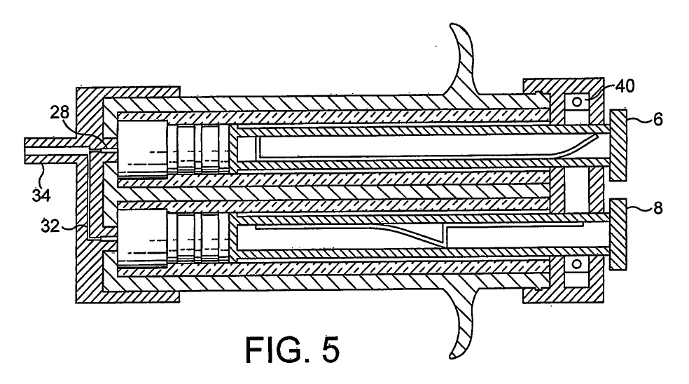

FIG. 5 shows the syringe after flushing.

Turning firstly to FIG. 1, there may be seen a cross-section through a syringe in accordance with the present invention. It may be seen that the syringe comprises two chambers in the form of glass vials 2,4 each being associated with a respective plunger 6,8. Each plunger 6,8 has a synthetic rubber bung 10,12 attached or over-moulded onto the end of the corresponding plunger. The upper vial 2 is intended in use to contain a lyophilised drug and thus the plunger bung 10 is designed to provide any necessary barrier properties and/or difference in pressure relative to ambient which might pertain to the required storage of the particular lyophilised drug in question.

The lower vial 4 is intended in use to contain a liquid diluent suitable for reconstituting the lyophilised drug in the upper chamber 2. Thus, the bung 12 on the diluent plunger 8 is capable not only of providing a liquid-tight seal, but also of providing a sufficient barrier against contamination of the sterilised diluent. In this embodiment the plungers 6,8 for the drug and diluent are coloured red and blue respectively for ease of identification but this is by no means essential.

At the opposite end of each vial 2,4, is provided a rubber septum 14,16. As with the rubber bungs 10,12, the rubber septums 14,16 provide the required barrier properties for the intended shelf life of the materials in the syringe. The two vials 2,4, are held in a two-part clam shell outer casing moulding 18 which also protects them from damage. The outer casing 18 incorporates two finger rests 20 which, as is conventionally known, allow a user to apply thumb pressure to the ends of the plungers 6,8.

The front end 18a of the outer moulding defines a pair of apertures 22,24 in alignment with the two respective septums 14,16 to provide access to them. The housing front end 18a is covered by a plastics front end moulding 26 which is mounted so as to be able to slide axially relative to the syringe housing 18. In the storage position shown in FIG. 1 it is offset towards the left—i.e. at the leftmost end of its travel, when viewed from FIG. 1.

The front end moulding 26 has a pair of steel needles 28,30 mounted inside which extend into the apertures 22,24 respectively in the front face of the syringe housing 18a. In an alternative embodiment (not shown) the needles can be integrally moulded from the same plastics material as the front cap. The two needles 28,30 are fluidly connected at their other ends by a conduit in the form of a narrow bore 32. Although not visible in the Figure, the narrow bore 32 has a tortuous configuration.

Also in fluid communication with the narrow bore 32 is the central bore of a nozzle 34 at the front end of the front end moulding 26. The nozzle 34 will in use be connected to an administration set such as a line set, cannula or hypodermic needle and thus defines the exit path 36 from the syringe. The nozzle 34 is of standard configuration in order to co-operate with commonly available administration sets. A nozzle cap 36 is provided over the nozzle 34 to maintain sterility of the syringe.

Attached to the rear end of the syringe housing 18 is another plastics moulding 38, although this could equally be integrally formed with the housing. The rear moulding 38 defines a void in which is provided a cam guiding shuttle 40 which is moveable in within the rear moulding 38 in a direction perpendicular to the plungers 6,8. The shuttle 40 has a pair of integrally moulded cam followers in the form of pins (not visible) which engage respectively in grooves defining cam tracks 42,44 in the shafts of the two plungers 6,8.

It will be seen that in FIG. 1, the shuttle 40 is in its upper position since the two followers are at the leftmost ends of the cam tracks 42,44. It should further be noted that although the upper cam follower is not prevented from moving down (i.e. towards the main axis of the syringe) by its cam track 42, it is prevented from doing so by virtue of being integrally formed with the shuttle 40 and therefore the lower cam follower which is prevented from moving in the aforementioned direction by the lower cam track 44. The result of this is that the plunger 6 cannot be depressed the user is unable to attempt to expel the lyophilised drug from the vial 2. Moreover, by preventing movement of the drug vial plunger 6, a vacuum may be maintained in the drug vial 2 which may be important for certain drugs.

During fabrication of the syringe shown in FIG. 1, the two clam shells of the front end moulding 26 and the needles 28 are assembled together by e.g. ultrasonic welding. The nozzle cap 36 is then added to this assembly. The front end assembly is then assembled onto one clam shell half of the syringe outer housing 18 along with the rear end moulding 38 and the shuttle 40.

The lower glass vial 4 and plunger 8 are fabricated and assembled and then filled with a suitable diluent before the rubber septum 16 is fitted. The diluent vial assembly is then mounted into the bottom half of the main syringe housing 18 to which the front and rear mouldings 26, 38 were attached. The whole assembly is then passed through a terminal sterilisation process, known per se.

In a separate part of the fabrication plant, the upper glass vial 2 (with plunger 6) is filled with the desired liquid drug and then passed through the stages, well known per se in the art, necessary to lyophilise the drug and fit the rubber septum 14.

The drug vial assembly 2,6,14 is assembled into the lower half of the syringe housing 18 and the upper half of the housing 18 added and secured to complete the assembly. Thus the diluent vial 4 and the rest of the syringe may be sterilised without harming the delicate lyophilised drug which may be prepared in its vial 2 in the normal way. This makes assembly of the syringe straightforward.

It will be appreciated by those skilled in the art that the syringe as shown in FIG. 1 may be stored for a long period of time since the lyophilised drug and the diluent are completely sealed within the two vials 2,4 respectively. Moreover, lyophilisation of the drug ensures its long term stability. Although the position of the shuttle 40 is not preventing depression of the diluent plunger 44, in practice such depression is prevented by hydraulic resistance of the diluent in the vial 4 since, as mentioned above, the vial is completely sealed.

When it is desired to use the drug by injecting it, the lyophilised drug must first be reconstituted. The first step in achieving this is shown in FIG. 2. As will be seen from a consideration of FIGS. 1 and 2, in FIG. 2 the front end moulding 26 has been slid onto the front end of the syringe housing 18a. This causes the needles 28,30 to pass through the apertures 22,24 in the front of the housing 18a and to pierce the septums 14,16. A fluid path between the two vials 2,4 is therefore provided via the two needles 28,30 and the narrow bore 32. It will be noted that the cap 36 is retained at this stage in order to ensure that the fluid paths 28-34 and the two vials 2,4 are maintained sterile and to ensure that the sole fluid path opened is between the two vials.

Since, in the described embodiment, the lyophilised drug is stored under vacuum in the drug vial 2, when the fluid path is opened as shown in FIG. 2, diluent from the diluent vial 4 will automatically be drawn through the narrow bore 32 into the drug vial 2. The reduction in pressure in the diluent vial 4 causes the diluent plunger 8 to move forwards as may be seen in FIG. 3. Depending upon the degree of vacuum in the drug vial 2, it may be necessary to apply some manual pressure to the plunger 8 to reach the position shown in FIG. 3. In an alternative embodiment (not shown) the drug is not stored under pressure and the configuration of the cam tracks 42,44 is different in order to allow the syringe to be stored with the drug plunger partially depressed and able to move out of the drug vial to accommodate diluent being injected in.

The tortuous narrow bore 32 ensures a high pressure drop across it. This means that under normal manual pressure on the diluent plunger 8, diluent will exit into the drug vial 2 at a dribble—i.e. such a low liquid velocity that jetting does not occur and potentially harmful foaming or shearing are avoided. Mixing of the diluent and the lyophilised drug in the drug vial 2 effects reconstitution of the drug ready for use.

It may further be seen from FIG. 3 that the forward movement of the diluent plunger 8 and the downwardly curving shape of the corresponding part of its cam track 44 causes the shuttle 40 to move from its original upper position to the lower position. The upwardly kinked shape of the cam track 44 at this point prevents any further forward movement of the diluent plunger 8. A proportion of the diluent will therefore remain in the diluent vial 4.

It will further be appreciated that since in FIG. 3 the shuttle 40 has moved downwards, this will move the cam follower in the cam track 42 of the drug plunger 6 thereby enabling it to be depressed when required. However, this will not be possible until the correct quantity of diluent has been dispersed.

Once reconstitution of the drug has taken place, the cap 36 on the nozzle 34 may be removed and the nozzle 34 attached to a cannula or butterfly introducer set. The drug plunger 6 is then actuated a short way to purge the system of air as will be well known to the user. The user will then typically insert the hypodermic needle into the patient (which could of course be the user themself), carrying out a vein check to ensure that the needle is correctly inserted or not in the blood stream as appropriate. The user may then depress the drug plunger 6 fully in order to inject the reconstituted drug into the patient. During this part of the operation, the syringe is used exactly as a conventional syringe would be.

The skilled person will appreciate that although the fluid path between the vials 2,4 via the narrow bore 32 remains open during the injection procedure, in practice the difference in pressure between the narrow bore 32 and the exit path through the nozzle 34 will ensure that substantially none of the drug is diverted into the narrow bore 32 or the diluent vial 4. If necessary, this can be further ensured by providing the diluent bung 12 with barbed-like projections in order to increase its resistance to moving back along the vial 4.

Turning now to the shuttle 40, it will be seen that after full actuation of the drug plunger 6, the upward curve at the end of its cam track 42 causes the shuttle 40 again to move to its upper position. This permits further actuation of the diluent plunger 8 as may be appreciated by considering the shape of its cam track 44.

The final step in the procedure is shown in FIG. 5. In this Figure, it may be seen that the diluent plunger 6 has also been fully actuated as is permitted by the upward position of the shuttle 40. This flushes all the internal fluid paths 30,32,34 of any remaining drug using the remainder of the diluent. The only part of the system which will not be flushed is the drug needle 28, but it will be appreciated by those skilled in the art that the volume of the needle 28 is extremely low and at least an order of magnitude less than the volume of a standard subcutaneous cannula. The difference is even more pronounced when compared with a butterfly introducer. Thus, it may be ensured that the patient receives substantially all of the intended dose.

It will be seen from the foregoing description that at least a preferred embodiment of the invention provides a two-part syringe which can be used to store and subsequently reconstitute a lyophilised drug and thereafter inject it into a patient in an entirely conventional manner without the user ever having to come into contact either with the drug or the diluent, or indeed without having manually to pierce the septums with a needle.

More importantly, however, it will also be seen that the interlinking of the two plungers by the shuttle and respective cam tracks ensures that only the correct plunger may be actuated at the correct time. This makes administration of the drug by non-medical staff or self-administration by patients a greater possibility.

It will be appreciated by those skilled in the art that the embodiment described above is just one example of the application of the principles of the present invention and that many variations and modifications are possible within the scope of the invention. For example, the invention is not limited to the reconstitution of lyophilised drugs and indeed embodiments of the invention may find many different applications where it is desired to ensure the correct sequence of actuation of a pair of syringe plungers.

1. A two-part syringe comprising two chambers, each chamber having an associated plunger operable to eject fluid therefrom, said plungers being interlinked so as selectively to prevent movement of one of said plungers in its respective chamber dependent upon the position of the other plunger.

2. A syringe as claimed in claim 1 comprising a cam means is associated with the plungers.

3. A syringe as claimed in claim 2 wherein each plunger comprises a cam track cooperating with a common cam shuttle member comprising followers for each cam track.

4. A syringe as claimed in claim 3 wherein said cam shuttle is constrained to move within a void extending perpendicularly to the plungers.

5. A syringe as claimed in claim 1 for use in reconstituting and injecting a lyophilised drug, wherein said interlinking is configured to prevent injection of the drug until it has been reconstituted with diluent.

6. A syringe as claimed in claim 1 wherein the plungers are different in appearance.

7. A syringe as claimed in claim 1 arranged such that the two chambers can be placed selectively in fluid communication with one another.

8. A syringe as claimed in claim 7 comprising a communicating conduit which is movable into fluid communication with one or both of the chambers.

9. A syringe as claimed in claim 8 wherein said movement is arranged to breach a seal such as a septum or the like associated with one or both of the chambers.

10. A syringe as claimed in claim 9 wherein the communicating conduit comprises one or two needles for breaching said seal or seals.

11. A syringe as claimed in claim 8 wherein said communicating conduit comprises a tortuous or complex fluid path such that liquid forced therethrough under ordinary manual pressure on one of the plungers is caused to exit the conduit substantially without jetting.

12. A syringe as claimed in claim 8 wherein said communicating conduit is provided on a separate support member which is preferably slidable towards or away from the chambers.

13. A syringe as claimed in claim 12 wherein said separate support member is made from plastics.

14. A syringe for reconstituting a lyophilised substance comprising a first chamber containing or for containing the lyophilised substance and a second chamber containing or for containing a diluent; the first and second chambers being arranged generally adjacent one another; the syringe further comprising a communicating conduit for fluidly connecting the two chambers, said fluid connection means being selectively engageable with the two chambers.

15. A syringe as claimed in claim 14 wherein said conduit is provided on a support member separate from the two chambers.

16. A syringe as claimed in claim 15 wherein said support member is such as to be slidable so as to bring the conduit into and out of engagement with the chambers.

17. A syringe as claimed in claim 14 wherein the two chambers have respective associated plungers which are interlinked to prevent activation of one depending upon the position of the other.

18. A syringe as claimed in claim 14 wherein the conduit has a tortuous configuration.
